(19) United States
(12) Patent Application Publication
(10) Pub. No.: US 2026/0047830 A1
(43) Pub. Date: Feb. 19, 2026

(54) ALIGNING UNIT AND ASSEMBLY METHOD FOR A MEDICAL INSTRUMENT

(71) Applicant: Aesculap AG, Tuttlingen (DE)

(72) Inventors: Andre Buerk, Villingen-Schwenningen (DE); Aaron Vogler, Leibertingen (DE)

(21) Appl. No.: 19/101,993

(22) PCT Filed: Aug. 3, 2023
(86) PCT No.: PCT/EP2023/071588

§ 371 Date: Feb. 7, 2025

(30) Foreign Application Priority Data
Aug. 9, 2022 (DE) 10 2022 119 981.9

(51) Int. Cl. A61B 17/00

(52) U.S. Cl. CPC A61B 17/00; A61B 2017/00469; A61B 2017/00526

(57) ABSTRACT

An alignment unit for a medical instrument includes a stator housing in which a drive unit, preferably in the form of a handpiece, can be accommodated, a threaded bolt with an external thread portion connecting the threaded bolt to the stator housing, and a carrier with an internal thread portion connecting the carrier to the stator housing. The external thread portion of the threaded bolt and the internal thread portion of the carrier are formed so as to be opposed to one another and are fixed by rotational movement in opposite directions such that the carrier and the threaded bolt are axially in contact with one another. The alignment unit can be assembled using an assembly method in which the threaded bolt is screwed into the stator housing.

### CROSS-REFERENCE TO RELATED APPLICATIONS

This application is the United States national stage entry of International Application No. PCT/EP2023/071588, filed on Aug. 3, 2023, and claims priority to German Application No. 10 2022 119 981.9, filed on Aug. 9, 2022. The contents of International Application No. PCT/EP2023/071588 and German Application No. 10 2022 119 981.9 are incorporated by reference herein in their entireties.

### FIELD

The present disclosure relates to an alignment unit for a medical instrument, in particular for a hand instrument. Furthermore, the present disclosure relates to an assembly method for the alignment unit.

### BACKGROUND

In the case of medical instruments in general and hand instruments in particular, it is often necessary to connect two shaft-like components, for example cylindrical/hollow shaft/hollow body/sleeve/tubular components, to one another, which can be done in a simple manner via a threaded connection.

One advantage of such threaded connections is that the two components to be connected can be adjusted to each other in an infinitely variable manner, so that, for radial alignment, they can either be screwed in further/screwed on or unscrewed to a predetermined rotational position in relation to each other over the length of the threaded connection. Radial alignment is particularly necessary if one of the two components has a groove, such as an insertion groove, or a protrusion, which must be arranged/aligned at a specific circumferential point due to external installation space conditions.

At the same time, this is also a disadvantage of such threaded connections, since they must be axially in contact with a counter bearing in order to be tightened with a predetermined torque and thus secured against unintentional loosening. Typically, axial abutment is achieved by completely screwing one of the two components to be connected into the other of the two components to be connected (or onto the other component) until it is in contact with it. However, if the components are screwed into/onto each other completely, radial alignment of the two components again is no longer possible.

### SUMMARY

The present disclosure is based on the task of reducing or avoiding disadvantages of the prior art. In particular, an alignment unit and an assembly method for mounting the alignment unit are to be provided in which two components can be simultaneously firmly connected to each other, in particular axially fixed in an arbitrary or non-fixed axial position, and radially aligned to each other. In addition, the alignment unit is to be compact and cost-effective, and easy to assemble.

The underlying problem of the present disclosure is solved by an alignment unit for a medical instrument, in particular a hand instrument, as well as by an assembly method for the alignment unit. Advantageous further developments will be described later in more detail.

To put it more precisely, the task is solved by an alignment unit for a medical instrument, in particular a hand instrument which comprises a stator housing, a threaded bolt and a carrier. A drive unit, preferably in the form of a (motorized) handpiece, can be accommodated in the stator housing. Preferably, the stator housing may comprise a (distal) substantially tubular end portion. The threaded bolt comprises an external thread portion connecting the threaded bolt to the stator housing (i.e. an internal thread in the stator housing). Preferably, the threaded bolt may comprise a (proximal) substantially tubular end portion, on the outside of which the external thread portion is formed. The external thread portion of the threaded bolt may be screwed into the end portion of the stator housing. The carrier comprises an internal thread portion connecting the carrier to the stator housing (i.e. an external thread in the stator housing). Preferably, the carrier may comprise a (proximal) substantially tubular end portion, on the inside of which the internal thread portion is formed. The internal thread portion of the carrier may be screwed onto the end portion of the stator housing.

In other words, the alignment unit comprises the stator housing, the threaded bolt and the carrier, wherein both the threaded bolt and the carrier each are attached to the stator housing, in particular to the distal end portion of the stator housing, via a threaded connection or are screwed on from the proximal side. In this case, the threaded bolt preferably may be mounted to the stator housing on the inside and the carrier on the outside. Alternatively, the threaded bolt could also be mounted to the outside of the stator housing and the carrier to the inside. This means that the threaded connections of the threaded bolt and the carrier are complementary (i.e. formed on the inside and on the outside), but cannot be/are not screwed together, but, respectively, to a corresponding threaded connection on the stator housing.

According to the present disclosure, the external thread portion of the threaded bolt and the internal thread portion of the carrier are configured so as to oppose each other. This means, for example, that the threaded bolt comprises a right-hand thread and the carrier comprises a left-hand thread, or that the threaded bolt comprises a left-hand thread and the carrier comprises a right-hand thread. The threaded bolt and the carrier are fixed by counter-rotation/locking such that the carrier and the threaded bolt are in axial contact with each other, in particular are braced against each other. This has the advantage that the threaded bolt would have to be turned in in a different direction than the carrier to loosen it, so that a frictional moment created through axial contact between the threaded bolt and the carrier counteracts any unintentional loosening of the two threaded connections. At the same time the infinitely variable adjustment is maintained over the length of the threaded connections and thus the possibility for radial alignment, since the axial position at which the threaded bolt and the carrier are in contact with each other is not fixed by the end of the thread, but can be set as desired/required by the screw penetration depth of the two threaded connections.

Accordingly, the task is solved in particular by the fact that both the threaded bolt and the carrier are screwed to the stator housing, respectively, via one thread connection/thread portion, the (external) thread portion of the threaded bolt, and the (internal) thread portion of the carrier being configured so as to be opposed to one another and being fixed/fixable by counter-rotation/locking such that the carrier and the threaded bolt axially are in contact with one another (at their stop surfaces) and in particular are braced against each other. As a result, the threaded bolt and the carrier can be fixed axially through bracing, without an axial position (determined, for example, by the end of the thread) and thus, also an alignment in a rotational position being fixed.

Preferably, the (internal) thread of the stator housing to which the threaded bolt can be screwed, and the (external) thread of the stator housing to which the carrier can be screwed, can be formed at least partially in the same axial portion of the stator housing. This means that the tubular (end) portion of the stator housing can be screwed or is screwed on the outside to one component, in this case the carrier, and on the inside to the other component, in this case the threaded bolt. In other words, the threaded connections between the stator housing and the threaded bolt and between the stator housing and the carrier are arranged in a radially nested manner, at least partially, preferably over the entire axial/longitudinal extension of at least one of the (or both) threaded connections. In other words, the stator housing, the threaded bolt and the carrier are arranged in a radially nested manner at least in portions, with the threaded connections being arranged at least partially, preferably at least one of the (or both) threaded connections being arranged completely, in the radially nested area.

Preferably, the threaded connection between the stator housing and the threaded bolt and the threaded connection between the stator housing and the carrier have different diameters/nominal sizes. This allows for radial nesting.

According to a preferred embodiment, a first axial gap may be formed between the stator housing and the threaded bolt. This means in particular that the threaded bolt is not completely screwed into the stator housing and that further screwing is prevented by the axial contact of the threaded bolt with the stator housing. Thus, adjustability of the threaded bolt for radial alignment remains in both directions of rotation.

According to a preferred embodiment, a second axial gap may be formed between the carrier and the stator housing. This means in particular that the carrier is not completely screwed onto the stator housing, or that further screwing is prevented by the carrier being axially in contact with the stator housing. Thus, also with regard to the carrier adjustability for radial alignment in both directions of rotation remains.

According to a further development of the preferred embodiment, the alignment unit may comprise have an external housing. In the external housing, the stator housing can be accommodated. Furthermore, the external housing may be configured such that it covers the second axial gap between the carrier and the stator housing. This avoids disadvantages arising from the second gap created on the outside, such as, for example, pollutants from the outside being able to infiltrate the external housing or stator housing.

According to a preferred embodiment, the threaded bolt may comprise a form-fitting tool engagement geometry, in particular in the form of an internal hexagon, preferably formed on its inside, for introducing a torque via a first socket wrench. This allows the threaded bolt to be screwed into the stator housing, preferably from the outside using the first socket wrench, without the alignment unit having to be disassembled.

According to a preferred embodiment, the carrier may comprise a form-fitting tool engagement geometry, preferably formed on its outer side, for introducing a torque via a second socket wrench. This allows the carrier to be screwed onto the stator housing, preferably from the outside, via the second socket wrench, without the need to disassemble the alignment unit.

According to a preferred embodiment, the alignment unit may comprise a threaded sleeve formed separately from the stator housing and connected fixedly, in particular in a rotationally fixed and axially secured/axially fixed manner, to the stator housing. The threaded sleeve can form an internal thread portion, via which the external thread portion of the threaded bolt is connected to the stator housing. This means that the stator housing and the threaded bolt are indirectly fixed to each other via the threaded sleeve, which is designed as a separate insert part and is firmly connected to the stator housing. This has the advantage that the threaded connection between the stator housing and the threaded bolt can be manufactured particularly cost-effectively, since the internal thread has to be installed on the prefabricated threaded sleeve and not directly on the stator housing, which may be difficult to machine or to access.

According to a further development of the preferred embodiment, the threaded sleeve may comprise at least one radially protruding lug, in particular a lug that protrudes radially outwards, or alternatively a lug that protrudes radially inwards. For example, the threaded sleeve can have two lugs that are located opposite each other around the circumference. For the purpose of a connection with the stator housing that is secured in a form-fitting manner against rotation and/or secured in a form-fitting manner axially the lug can engage in the stator housing, in particular in a corresponding recess in the stator housing. This has the advantage that a firm connection between the threaded sleeve which can be executed as a standard component and the stator housing can be realized in a particularly simple way. In addition, in this way, a suitable material pairing can be chosen for the threaded connection, which is independent of the material choice of the stator housing.

According to an alternative embodiment, the stator housing may integrally form on the inside thereof an internal thread portion, via which the external thread portion of the threaded bolt is connected to the stator housing. This means that the internal thread portion can also be formed directly on the stator housing/integrally/in one piece with the stator housing. This can be particularly advantageous when the installation space is very limited, so that a separate insert part cannot be used.

According to a preferred embodiment, the alignment unit may comprise a rotor shaft that is connectable to the drive unit in a torque-transmitting manner. For example, via a torque-transmitting connection to the rotatable rotor shaft, the rotation of the rotor shaft can be converted directly into the rotation of a tool of the hand instrument or indirectly into a motion coupled thereto, such as an angular deflection, of the tool. The rotor shaft can protrude axially in sections into the threaded bolt, in particular into an axial region, in which the external thread portion of the threaded bolt, or the tool engagement geometry of the threaded bolt is formed and can have on its outside, in particular in the axially protruding region, a tool engagement geometry, in particular in the form of an external hexagon, for introducing a torque via a socket wrench, preferably via the first socket wrench. That is, the tool engagement geometry of the rotor shaft and the threaded bolt preferably are arranged radially nested in one another, so that, with a corresponding design of the socket wrench, they can be turned by the same socket wrench and, if necessary, simultaneously by the socket wrench. This has the advantage that simultaneously, a radial alignment between the rotor shaft and the threaded bolt and a rotationally fixed fixation of both components can be obtained respectively via the socket wrench.

According to a further development of the preferred embodiment, the rotor shaft may comprise a funnel-shaped tapered outlet of the tool engagement geometry. That is, the tool engagement geometry merges with the outer diameter of the rotor shaft via radially tapered surfaces. This has the advantage that it is easier to attach the socket wrench. In addition, this ensures an automatic alignment of the rotor shaft with the socket wrench, so that the socket wrench can be used even in the area of the rotor shaft that is difficult to see due to its internal arrangement.

According to a further development of the preferred embodiment, the rotor shaft, in particular at its distal end portion, may comprise an internal thread portion for receiving an axial securing element that can be screwed onto the internal thread portion. For example, the axial securing element can be formed as a threaded sleeve which has on the inside thereof a screw-in geometry, in particular in the form of an internal hexagon. Due to the fact that a torque can be applied to the rotor shaft via the first socket wrench, the rotor shaft can be held in place using the first socket wrench when the axial locking element is screwed into the internal thread portion of the rotor shaft. This allows the rotor shaft to be secured against rotation from outside, and the axial locking element, which in turn preferably axially secures a hexagonal insert, to be exchanged without having to disassemble the alignment unit. This makes it easy to replace parts that are prone to wear, such as the hexagonal insert, within the rotor shaft.

According to a preferred embodiment, the alignment unit may comprise a first socket wrench for introducing the torque into the tool engagement geometry of the threaded bolt and for introducing the torque into the tool engagement geometry of the rotor shaft. The first socket wrench can have a hollow cylindrical/hollow body shaped engagement portion on the outside of which is formed a first counter-portion, in particular in the form of an external hexagon, which is complementary to the tool engagement geometry of the threaded bolt, and on the inside of which is formed a second counter-portion, in particular in the form of an internal hexagon, which is complementary to the tool engagement geometry of the rotor shaft. Thus, the first socket wrench can be utilized both for introducing the torque into the threaded bolt and for introducing the torque to the rotor shaft and, respectively, for holding the rotor shaft.

According to a further development of the preferred embodiment, the first counter-portion and the second counter-portion may preferably be of the same shape, i.e. in particular both as a hexagon, in particular with the same radial alignment. This has the advantage that the engagement portion can be formed with a comparatively small wall thickness, so that it is space-saving and can also be inserted into a small radial gap between the rotor shaft and the threaded bolt.

According to a further embodiment of the preferred embodiment, the first socket may include an axial stop surface arranged such that both the first counter-portion is in engagement with the tool engagement geometry of the threaded bolt, and the second counter-portion is in engagement with the tool-engagement geometry of the rotor shaft when the axial stop surface axially abuts the threaded bolt or the rotor shaft, i.e. when the first socket wrench is in socket engagement/fully inserted. This limits the insertion depth of the first socket wrench. At the same time, when the first socket wrench is fully inserted, it can be ensured that rotational coupling between the rotor shaft and the threaded bolt via the first socket wrench is achieved, so that the rotor shaft and the threaded bolt can be easily fixed/held in place and the torque can be introduced at the same time.

The object of the present disclosure is also solved by a medical instrument, in particular a hand instrument, which comprises the alignment unit.

Furthermore, the object of the present disclosure is solved by an assembly method for assembling the alignment unit, wherein in a first step, the threaded bolt is screwed into the stator housing until the threaded bolt is in axial contact with the stator housing, in a second step, the carrier is screwed onto the stator housing until the carrier is in axial contact with the threaded bolt, in a third step, the carrier is unscrewed from the stator housing until the carrier has a predetermined radial alignment relative to the stator housing, in a fourth step, the threaded bolt is unscrewed from the stator housing until the threaded bolt is in axial contact with the carrier, and in a fifth step, the carrier and the threaded bolt are twisted in opposite rotational directions to one another.

According to a preferred embodiment of the assembly method, in a step preceding the first step, the threaded sleeve may be inserted in the stator housing in a non-rotatable and axially fixed manner, the threaded bolt, in the first step, being screwed into the threaded sleeve for fastening in the stator housing.

According to a preferred embodiment of the assembly method, in the fourth step, a predetermined angular offset, for example of 10 degrees, may be maintained with respect to the predetermined radial alignment, e.g. by means of an aid, so that the predetermined angular offset can be compensated for again by twisting in the opposite direction, i.e. by applying a predetermined tightening torque during locking, and after the fifth step, the predetermined radial alignment is precisely provided.

In other words, the present disclosure relates to an alignment unit for radially aligning a carrier relative to a stator housing. In this case, a thread is used to provide infinitely variable adjustment. The threaded connection can be adjusted from the outside, so that no disassembly is required for adjustment. In addition, a left-hand thread is employed to secure the connection against loosening. In particular, a threaded sleeve can be inserted into the stator housing in a non-rotating manner, for example using two lateral lugs. A threaded bolt can then be screwed completely into the threaded sleeve. A carrier can now be screwed completely onto the stator housing until an axial stop surface is reached on the threaded bolt. For radial alignment of the carrier-especially if a circumferential insertion groove is not in the desired/correct position-the carrier can then be unscrewed until the desired alignment of the carrier is achieved. Subsequently, the threaded bolt is unscrewed/screwed in the distal direction until the axial stop surface on the carrier is reached. This creates an (axial) gap between the carrier and the stator housing, which, however, may be covered by an external housing. The final fixation can be performed by turning the threaded bolt and the carrier in opposite directions, i.e. by locking.

Preferably, a torque may be introduced to the threaded bolt by a hex wrench and countered on the carrier using an external socket wrench. This allows a slight twisting when tightening to be compensated for by correspondingly holding an angle. For example, a twisting of 10 degrees predefined by auxiliary means can be realized, which compensates when the predetermined torque is reached, so that a 0 degree position is achieved.

Furthermore, the alignment unit may be used for a quick-change function of a hexagonal insert received in the rotor shaft. In this case, the rotor shaft can be secured against rotation by the two hexagons, i.e. the internal hexagon on the threaded bolt and the external hexagon on the rotor shaft, and the hexagonal insert can be replaced without the handpiece/alignment unit/instrument having to be disassembled. In this case, the hexagonal geometry of the hexagonal insert serves to transfer a torque from the rotor shaft to a tool or the like of the instrument and is preferably configured as a separate insert part. This separate insert part, which is subject to wear, can be replaced easily by inserting the socket wrench into the alignment unit and onto the rotor shaft to fix the rotor shaft. The hexagonal insert is typically secured axially by a threaded sleeve with a hexagon socket that can be loosened via its hexagon socket while the rotor shaft is held firmly.

### BRIEF DESCRIPTION OF THE DRAWINGS

FIG. 1 is a longitudinal section of an alignment unit according to the present disclosure in a fully assembled position;

FIGS. 2 to 6 show the alignment unit at different times of an assembly process according to the disclosure; and

FIGS. 7-9 show various views of the alignment unit and a socket wrench for introducing torque to a threaded bolt and a rotor shaft of the alignment unit.

### DETAILED DESCRIPTION

A preferred embodiment of the present disclosure is described below, based on the associated figures.

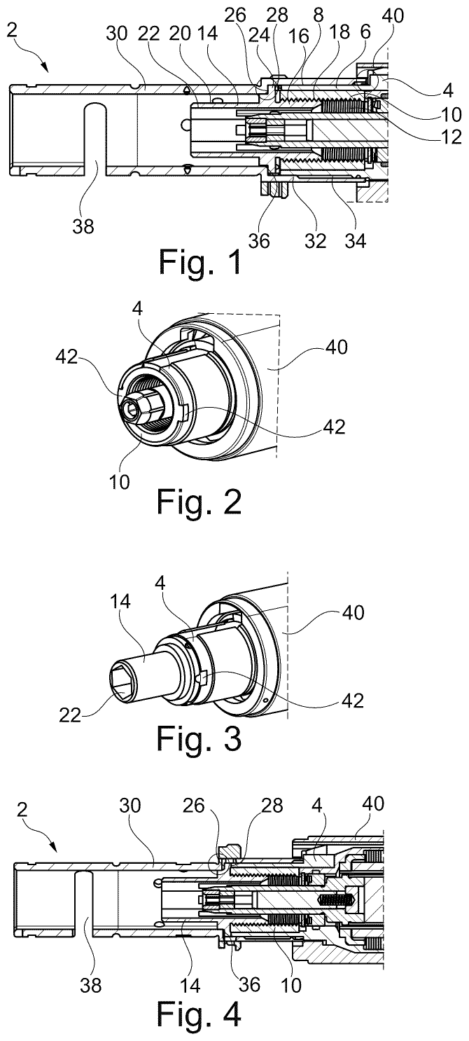

FIG. 1 shows an alignment unit 2 for a medical instrument, in particular a hand instrument, according to the present disclosure in a fully assembled position.

The alignment unit 2 comprises a stator housing 4. In particular, the stator housing 4 in particular forms a proximal component of the medical instrument. A drive unit, preferably in the form of a (motorized) handpiece (not shown), can be accommodated in the stator housing 4. The stator housing 4 forms a cavity for receiving the drive unit and other components of the medical instrument. At its distal end, the stator housing 4 has an essentially tubular end portion/sleeve portion 6. The end portion 6 has an external thread portion 8 on its outside. On an inner side of the end portion 6 a threaded sleeve 10 formed separately from the stator housing 4 is received, on which an internal thread portion 12 is formed.

The alignment unit 2 comprises a threaded bolt 14. The threaded bolt 14 is arranged distally of the stator housing 4. The threaded bolt 14 is hollow/has a central passage. At its proximal end, the threaded bolt 14 has an essentially tubular end portion 16. The end portion 16 has an external thread portion 18 on its outside. The external thread portion 18 of the threaded bolt 14 is formed so as to be complementary to the internal thread portion 12 of threaded sleeve 10. The threaded bolt 14 is screwed with/screwed into the stator housing 4 via the external thread portion 18. At its distal end, the threaded bolt 14 has a substantially tubular end portion 20. The end portion 20 has a tool engagement geometry 22 on its inside, via which a torque (form-fit) can be introduced into the threaded bolt 14 (from the distal side). The tool engagement geometry 22 is in the form of an internal hexagon. The threaded bolt 14 has a radially outwardly projecting protrusion 24. In the illustrated embodiment, the protrusion 24 is in the form of a circumferential flange. A first axial end surface 26 is formed on the protrusion 24 in the distal direction (on an axial side facing away from the stator housing 4). A second axial end surface 28 is formed on the protrusion 24 in the proximal direction (on an axial side facing the stator housing 4). In the fully assembled state, a first axial gap is formed between the second axial end surface 28 and the stator housing 4 and, respectively, the threaded sleeve 10, i.e. the threaded bolt 14 is not screwed into the threaded sleeve 10 completely/up to the stop.

The alignment unit 2 comprises a carrier 30. The carrier 30 is arranged distally of the stator housing 4. The carrier 30 is hollow/has a central passage. The carrier 30 has a substantially tubular end portion 32 at its proximal end. The end portion 32 has an internal thread portion 34 on its inside. The internal thread portion 34 of the carrier 30 is formed so as to be complementary to the external thread portion 8 of the stator housing 4. The carrier 30 is screwed to the stator housing 4/screwed onto the stator housing 4 via the internal thread portion 34. The carrier 30 has a tool engagement geometry (not explicitly shown) on its outside, via which a torque (form-fit) can be introduced into the carrier 30 (from the outside). Distally of the internal thread portion 34, the carrier 30 tapers via a radial step, by means of which an axial contact surface 36 is formed. In the assembled state, the axial contact surface 36 is in contact with the first axial end surface 26 of the threaded bolt 14. In the fully assembled state, a second axial gap is formed between the end portion 32 and the stator housing 4, i.e. the carrier 30 is not fully screwed/screwed up to the stop onto the stator housing 4. The carrier 30 has an insertion groove 38, which is formed on the circumference of the carrier 30 (here as a through-hole) and extends in the axial direction of the carrier 30. In the mounted state of the alignment unit 2, the insertion groove 38 is arranged in a predetermined alignment/circumferential position/rotational position (in the illustrated embodiment, so that it is arranged at the bottom/in a 0° position).

Preferably, the alignment unit 2 may comprise an external housing 40, in which the stator housing 4 is arranged, which is configured such that it covers the second axial gap between the carrier 30 and the stator housing 4.

According to the present disclosure, the external thread portion 18 of the threaded bolt 14 and the internal thread portion 34 of the carrier 30 are formed so as to be opposed to each other. This means that the threaded bolt 14 and the carrier 30 have to be turned in different rotational directions in order to be screwed on/in further or unscrewed/screwed out. Moreover, the threaded bolt 14 and the carrier 30 are fixed/locked by counter-rotation in such a way that the carrier 30 and the threaded bolt 14 are axially braced against each other.

An assembly method for assembling the alignment unit 2 is explained subsequently:

In a step preceding the assembly method, which is shown in FIG. 2, the threaded sleeve 10 is inserted into the stator housing 4. The threaded sleeve 10 has two radially outwardly projecting lugs 42 that engage in corresponding recesses in the stator housing 4, so that the threaded sleeve 10 is connected to the stator housing 4 in a rotation-securing manner and in an axially fixed manner.

In the first step of the assembly method, which is shown in FIG. 3, the threaded bolt 14 is screwed into the stator housing 4 (and, respectively, into the threaded sleeve 10). The threaded bolt 14 is fully screwed into the stator housing 4, i.e. until the threaded bolt 14 is in axial contact with the stator housing 4 (and the threaded sleeve 10, respectively). The torque is introduced into the threaded bolt 14 in particular via the tool engagement geometry 22 in the form of the internal hexagon.

In a second step of the assembly method, which is shown in FIG. 4, the carrier 30 is screwed onto the stator housing 4. The carrier 30 is fully screwed onto the stator housing 4, i.e. until the axial contact surface 36 is in contact with the first axial end surface 26. The torque is introduced into the carrier 30 in particular via the external tool engagement geometry (not explicitly shown) of the carrier 30.

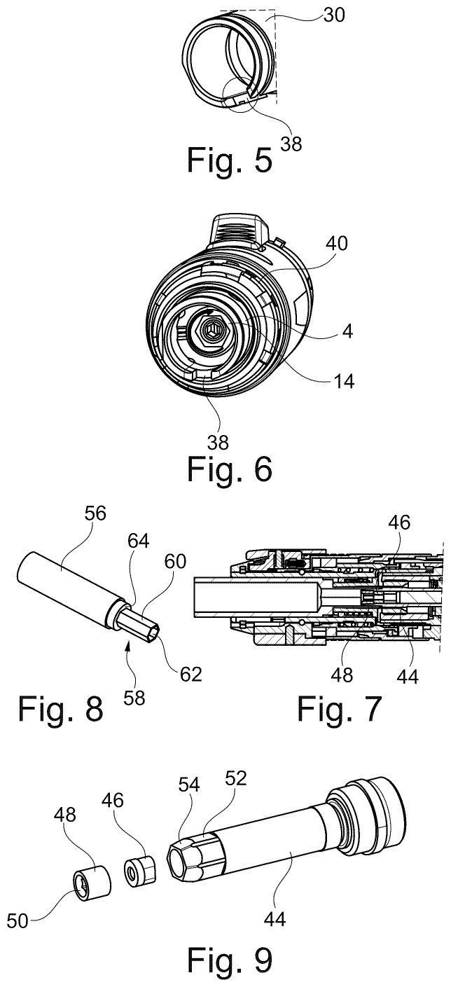

When the carrier 30 was fully screwed on, the insertion groove 38 of the carrier 30 as a rule is not in the desired radial alignment (see FIG. 5). Should this be the case, the carrier 30 is unscrewed from the stator housing 4 in a third step of the assembly method until the carrier 30 (in particular the insertion groove 38) has the predetermined radial alignment relative to the stator housing 4 (see FIG. 1). Subsequently, in a fourth step of the assembly method, the threaded bolt 14 is unscrewed from the stator housing 4 until the threaded bolt 14 with its first end surface 26 axially is in contact with the axial contact surface of the carrier 30 (see FIG. 6 and FIG. 1).

In a fifth step of the assembly method, the carrier 30 and the threaded bolt 14 are twisted/locked in opposite directions of rotation to each other, so that they are tightened/braced against each other with a predetermined torque.

In this case, a slight twisting when tightening/locking can be compensated for by correspondingly holding an angle, for example by means of an aid, whereby the angle is equalized when the predetermined torque is reached.

According to a further aspect of the present disclosure, the alignment unit 2 comprises a rotor shaft 44 that is connectable to the drive unit in a torque-transmitting manner (see FIGS. 7 to 9). The rotor shaft 44 protrudes axially in sections into the threaded bolt 14, in particular into an axial region in which the external thread portion 18 of the threaded bolt 14 or the tool engagement geometry 22 of the threaded bolt 14 is formed.

For example, via a torque-transmitting connection to the rotor shaft 44 drivable by rotation, the rotation of rotor shaft 44 can be converted directly into a rotation of a tool (not shown) of the instrument/hand instrument or indirectly into a coupled motion, such as an angular deflection, of the tool.

For this purpose, a hexagonal insert 46 is received in the rotor shaft 44 in a torque-proof manner, via which the rotation of the rotor shaft 44 can be further transmitted. The hexagonal insert 46 is formed as an insert part and has an internal hexagon on its inside for torque transfer. To secure the hexagonal insert 46 axially, a locking element in the form of a threaded sleeve 48 moreover is accommodated in the rotor shaft 44, which axially is in contact with the hexagonal insert 46 and thus prevents the hexagonal insert 46 from falling out of the rotor shaft.

The threaded sleeve 48 comprises an external thread on its outside, by means of which it is screwed to a correspondingly designed internal thread on the inside of the rotor shaft 44. To introduce a torque into the threaded sleeve 48, the threaded sleeve 48 has a hexagon socket 50.

The rotor shaft 44 comprises on its outside, in particular in the region that protrudes axially into the threaded bolt 14, a tool engagement geometry 52, in particular in the form of an external hexagon, for introducing a torque. The rotor shaft 44 preferably has a funnel-shaped tapering end 54 of the tool engagement geometry 52, approximately in the form of radially sloping surfaces of the external hexagon.

Furthermore, the alignment unit 2 comprises a socket wrench 56. The socket wrench 56 serves to initiate the torque in the tool engagement geometry 22 of the threaded bolt 14 and to initiate the torque in the tool engagement geometry 52 of the rotor shaft 44. The socket wrench 56 has a hollow cylindrical/hollow body-shaped engagement portion 58, on the outside of which a first counter-section 60, in particular in the form of an external hexagon, is formed which is complementary to the tool engagement geometry 22 of the threaded bolt 14, and on the inside of which a second counter-section 62, in particular in the form of an internal hexagon, is formed which is complementary to the tool engagement geometry 52 of the rotor shaft 44.

Preferably, the first counter-section 60 and the second counter-section 62 may be of the same shape, i.e. in particular both as a hexagon, in particular with the same radial alignment. Furthermore, the socket wrench 56 can have an axial end surface 64, which is arranged such that both the first counter-section 60 is in engagement with the internal hexagon of the threaded bolt 14, and the second counter-section 62 is in engagement with the external hexagon 52 of the rotor shaft 44, when the axial end surface 64 is in axial contact with the threaded bolt 14, i.e. when it is fully inserted (see FIG. 7).

### List of Reference Signs

2 Alignment unit
4 Stator housing
6 Tubular end portion
8 External thread portion
10 Threaded sleeve
12 Internal thread portion
14 Threaded bolt
16 Tubular end portion
18 External thread portion
20 Tubular end portion
22 Tool engagement geometry
24 Protrusion
26 First axial end surface
28 Second axial end surface
30 Carrier
32 Tubular end portion
34 Internal thread portion
36 Axial contact surface
38 Insertion groove
40 External housing
42 Radially protruding lug
44 Rotor shaft
46 Hexagonal insert
48 Threaded sleeve
50 Internal hexagon
52 Tool engagement geometry
54 Funnel-shaped tapering end
56 First socket wrench
58 Hollow body-shaped engagement portion
60 First counter-section
62 Second counter-section
64 Axial end surface

1-10. (canceled)

11. An alignment unit for a medical instrument, the alignment unit configured for a radial alignment of two components to be screwed on up to a mutual axial contact, the alignment unit comprising:
a stator housing for receiving a drive unit, the stator housing being tubular;
a threaded bolt comprising an external thread portion that connects to the stator housing; and
a carrier comprising an internal thread portion that connects to the stator housing, the carrier being tubular;
the stator housing, the threaded bolt and the carrier having a common longitudinal axis, and
the external thread portion and the internal thread portion being formed so as to oppose each other and being fixed by rotational movement in opposite directions such that the carrier and the threaded bolt are axially in contact with one another.

12. The alignment unit according to claim 11, wherein a first axial gap is formed between the stator housing and the threaded bolt.

13. The alignment unit according to claim 11, wherein:
a second axial gap is formed between the carrier and the stator housing, and
the alignment unit comprises an external housing for receiving the stator housing and for covering the second axial gap between the carrier and the stator housing.

14. The alignment unit according to claim 11, wherein the threaded bolt comprises a first tool engagement geometry for introducing a torque via a first socket wrench.

15. The alignment unit according to claim 14, wherein the first tool engagement geometry is formed on an inside of the threaded bolt.

16. The alignment unit according to claim 14, wherein the first tool engagement geometry is an internal hexagon.

17. The alignment unit according to claim 14, further comprising a rotor shaft connectable to the drive unit in a torque-transmitting manner, axially protruding in sections into the threaded bolt, the rotor shaft comprising a third tool engagement geometry on an outside of the rotor shaft, the third tool engagement geometry configured for introducing a torque via a socket wrench.

18. The alignment unit according to claim 17, wherein the third tool engagement geometry is an external hexagon.

19. The alignment unit according to claim 17, wherein the rotor shaft comprises a funnel-like tapering end of the third tool engagement geometry.

20. The alignment unit according to claim 17, further comprising a first socket wrench for introducing torque into the first tool engagement geometry of the threaded bolt and for introducing torque into the third tool engagement geometry of the rotor shaft, the first socket wrench comprising a hollow body-shaped engagement portion, forming a first counter-section on an outside the first socket wrench, the first counter-section being complementary to the first tool engagement geometry of the threaded bolt, and forming a second counter-section on an inside of the first socket wrench, the second counter-section being complementary to the third tool engagement geometry of the rotor shaft.

21. The alignment unit according to claim 20, wherein the first counter-section is an external hexagon and the second counter-section is an internal hexagon.

22. The alignment unit according to claim 11, wherein the carrier comprises a second tool-engagement geometry for introducing a torque via a second socket wrench.

23. The alignment unit according to claim 22, wherein the second tool-engagement geometry of the carrier is formed on an outside of the carrier.

24. The alignment unit according to claim 11, further comprising a threaded sleeve, the threaded sleeve being formed separately of the stator housing, being firmly connected to the stator housing, and forming an inner thread portion, the inner thread portion connecting the external thread portion of the threaded bolt to the stator housing.

25. The alignment unit according to claim 24, wherein the threaded sleeve comprises at least one radially protruding lug engaging into the stator housing for a connection with the stator housing secured against rotation in a form-fit manner and/or secured axially with the stator housing.

26. A method for assembling the alignment unit for the medical instrument according to claim 11, the method comprising the steps of:
screwing the threaded bolt into the stator housing until the threaded bolt is in axial contact with the stator housing;
screwing the carrier onto the stator housing until the carrier is in axial contact with the threaded bolt;
unscrewing the carrier from the stator housing until the carrier has a predetermined radial alignment relative to the stator housing;
screwing the threaded bolt out of the stator housing until the threaded bolt is in axial contact with the carrier, and
twisting the carrier and the threaded bolt in opposite directions to one another.
